(19) United States
(12) Patent Application Publication
(10) Pub. No.: US 2016/0302730 A1
(43) Pub. Date: Oct. 20, 2016

(54) GRIPPING-DETECTION DEVICE

(71) Applicant: HONDA MOTOR CO., LTD., Tokyo (JP)

(72) Inventor: Shotaro Odate, Wako-shi (JP)

(73) Assignee: HONDA MOTOR CO., LTD., Tokyo (JP)

(21) Appl. No.: 15/099,143

(22) Filed: Apr. 14, 2016

(30) Foreign Application Priority Data
Apr. 15, 2015 (JP) 2015-083417

(51) Int. Cl. A61B 5/00; A61B 5/0245; B60Q 9/00

(52) U.S. Cl. CPC A61B 5/6893; B60Q 9/00; A61B 5/0245; A61B 5/7203; A61B 5/6843; A61B 2560/0468; A61B 2562/0214; A61B 2503/12

(57) ABSTRACT

A gripping-detection device includes a first electrode and a second electrode provided on a steering wheel of a vehicle; a heartbeat detection unit that detects the heartbeat of a driver that is in contact with the first electrode and the second electrode based on a voltage pattern obtained from the first electrode and the second electrode; a first sensor and a second sensor that detect an electrostatic capacitance in the steering wheel; and a gripping state determination unit that determines a gripping state of the steering wheel of the driver based on the electrostatic capacitance detected by the electrostatic capacitance detection unit and a voltage pattern of the first electrode and the second electrode detected by the heartbeat detection unit.

### CROSS REFERENCES TO RELATED APPLICATIONS

The present application claims priority under 35 U.S.C. §119 to Japanese Patent Application No. 2015-083417, filed Apr. 15, 2015, entitled “Gripping-Detection Device.” The contents of this application are incorporated herein by reference in their entirety.

### BACKGROUND

1. Field

The present disclosure relates to a gripping-detection device.

2. Description of the Related Art

The related art discloses an automatic driving device that uses a pressure-sensitive contact sensor, a sweat sensor, a steering torque sensor, and the like provided on the steering wheel of a vehicle to detect whether or not a driver is in contact with a steering wheel and determine whether or not to apply automatic lane-keeping control based on the detection result (see, for example, Japanese Unexamined Patent Application Publication No. 2008-273521).

According to the automatic driving device of the related art described above, there is a problem that whether or not one or both of the driver's hands are actually in contact with the steering wheel cannot be accurately detected by the mere use of the pressure-sensitive contact sensor, the sweat sensor, the steering torque sensor, and the like.

### SUMMARY

In view of the above background, the present application describes a gripping-detection device that enables accurate detection as to whether or not one or both of driver's hands is in contact with a steering wheel.

(1) A gripping-detection device according to first aspect of the embodiment includes a pair of electrodes (for example, a first electrode (L) 13L and a second electrode (R) 13R in the embodiment) provided on a steering wheel (for example, a steering wheel 22 in the embodiment) of a vehicle; a heartbeat detection unit (for example, a heartbeat detection unit 14 in the embodiment) that detects the heartbeat of a driver in contact with the pair of electrodes based on a voltage pattern obtained from the pair of electrodes; an electrostatic capacitance detection unit (for example, a first sensor (L) 11L and a second sensor (R) 11R, and an electrostatic capacitance detection unit 12 in the embodiment) provided in the steering wheel near to where the pair of electrodes are arranged and detects electrostatic capacitance; and a gripping state determination unit (for example, a gripping state determination unit 15 in the embodiment) that determines a gripping state of the steering wheel of the driver based on the electrostatic capacitance detected by the electrostatic capacitance detection unit and a voltage pattern of the pair of electrodes detected by the heartbeat detection unit.

(2) In the gripping-detection device of (1) described above, the heartbeat detection unit may start detecting the heartbeat of the driver when an electrostatic capacitance that is greater than or equal to a predetermined value is detected by the electrostatic capacitance unit.

(3) In the gripping-detection device of (2) described above, when the electrostatic capacitance that is greater than or equal to a predetermined value is detected by the electrostatic capacitance unit and the heartbeat of the driver is detected by the heartbeat detection unit, the gripping state determination unit may determine that the driver is gripping the steering wheel with both hands.

(4) In the gripping-detection device of (2) described above, when the electrostatic capacitance that is greater than or equal to a predetermined value is detected by the electrostatic capacitance unit, and no heartbeat of the driver is detected and a noise that is greater than or equal to a predetermined amount is detected in the voltage pattern by the heartbeat detection unit, the gripping state determination unit may determine that the driver is gripping the steering wheel with one hand.

(5) In the gripping-detection device of (2) described above, when the electrostatic capacitance that is greater than or equal to a predetermined value is detected by the electrostatic capacitance unit, and no heartbeat of the driver is detected and a noise that is greater than or equal to a lower limit threshold and less than a predetermined amount is detected in the voltage pattern by the heartbeat detection unit, the gripping state determination unit may determine that a foreign material is in contact with the steering wheel.

(6) In the gripping-detection device of (2) described above, when the electrostatic capacitance that is greater than or equal to a predetermined value is detected by the electrostatic capacitance unit, and no heartbeat of the driver is detected and a noise that is less than a lower limit threshold is detected in the voltage pattern by the heartbeat detection unit, the gripping state determination unit may determine that the driver is gripping the steering wheel with a hand covered by a glove.

According to the gripping-detection device of the aspect (1) described above, the combined use of the detected electrostatic capacitance and the voltage pattern of the pair of electrodes in the steering wheel enables accurate detection of a gripping state of the driver.

Moreover, in the case of (2) described above, first, the likelihood of the driver being in contact with the steering wheel can be determined by detecting the electrostatic capacitance. Then, when the driver is likely to be in contact with the steering wheel, particular conditions of a gripping state can be determined based on the voltage pattern of the pair of electrodes. This enables a simplified determination process and therefore faster determination compared to the case where, for example, the determination is made based on a voltage pattern of a pair of electrodes prior to detection of an electrostatic capacitance.

Moreover, in the case of (3) or (4) described above, a gripping state of the driver can be determined with high accuracy.

Moreover, in the case of (5) or (6) described above, a state that is different from a state where the steering wheel is gripped by both hands or one hand of the driver can be determined.

### BRIEF DESCRIPTION OF THE DRAWINGS

FIG. 1 is a block diagram of a functional configuration of a gripping-detection device according to an embodiment of the present disclosure.

FIG. 2 is a diagram illustrating first and second sensors and first and second electrodes provided to a steering wheel of the gripping-detection device according to the embodiment of the present disclosure.

FIG. 3 is a configuration diagram of an electrostatic capacitance detection unit of the gripping-detection device according to the embodiment of the present disclosure.

FIG. 4 is a configuration diagram of a heartbeat detection unit of the gripping-detection device according to the embodiment of the present disclosure.

FIG. 5 is a diagram illustrating a relationship between a voltage pattern detected by the heartbeat detection unit and an object (a hand or a foreign material) that is in contact with the first electrode and the second electrode of the gripping-detection device according to the embodiment of the present disclosure.

FIG. 6 is a flowchart illustrating a process of the gripping-detection device according to the embodiment of the present disclosure.

FIG. 7 is a diagram illustrating a relationship between each waveform of an output voltage output from a reception selector circuit of the electrostatic capacitance detection unit and an object (a hands or a glove) that is in contact with the first sensor and the second sensor of the gripping-detection device according to the embodiment of the present disclosure.

### DESCRIPTION OF THE PREFERRED EMBODIMENTS

The embodiments of the present application will be described below with reference to the drawings. The word “unit” used in this application may mean a physical part or component of computer hardware including a controller, a processor, a memory, etc., which is configured to perform intended functions, as disclosed herein.

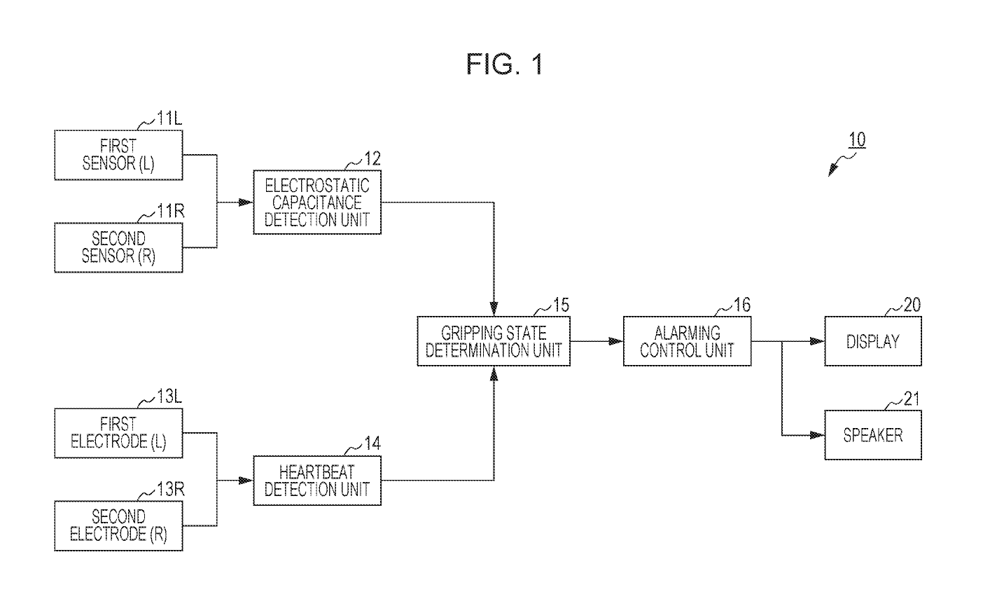

A gripping-detection device 10 according to the present embodiment is mounted in a vehicle. As illustrated in FIG. 1, the gripping-detection device 10 includes a first sensor (L) 11L and a second sensor (R) 11R, an electrostatic capacitance detection unit 12, a first electrode (L) 13L and a second electrode (R) 13R, a heartbeat detection unit 14, a dripping state determination unit 15, an alarming control unit 16, a display 20, and a speaker 21.

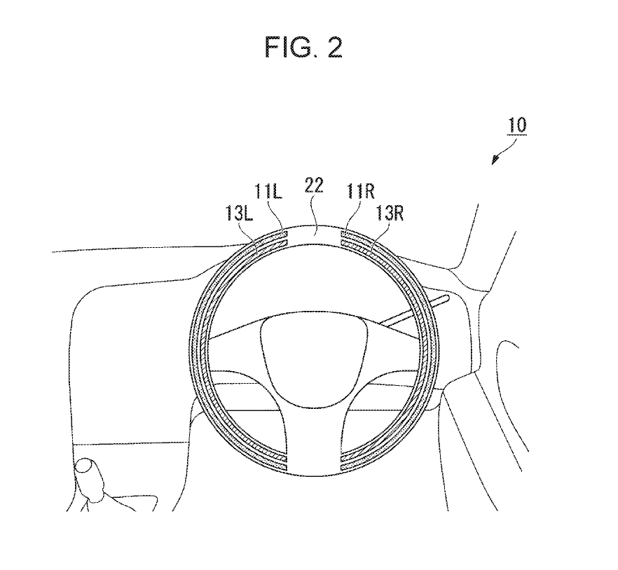

As illustrated in FIG. 2, the first sensor (L) 11L and the second sensor (R) 11R are provided inside a left part and a right part, respectively, of a steering wheel 22 in a straight-running position (a neutral position). The first sensor (L) 11L is provided in the left part in the steering wheel 22 in the neutral position, that is, a portion gripped by the driver's left hand. The second sensor (R) 11R is provided in the right part in the steering wheel 22 in the neutral position, that is, a portion gripped by the driver's right hand.

The first sensor (L) 11L and the second sensor (R) 11R may be formed of any electro-conductive material. For example, the first sensor (L) 11L and the second sensor (R) 11R are made of an electro-conductive material applied to an inner skin of the steering wheel 22, an electro-conductive leather material, or the like.

The first sensor (L) 11L and the second sensor (R) 11R are antenna electrodes in which electrostatic capacitance varies in accordance with distance and area with respect to a dielectric material such as in the case of a human body. The first sensor (L) 11L and the second sensor (R) 11R each emit a radio frequency signal (radio emission power) at a predetermined frequency supplied from the heartbeat detection unit 14. The first sensor (L) 11L and the second sensor (R) 11R form an electrostatic capacitance sensor with respect to a grounding portion such as a vehicle body, for example.

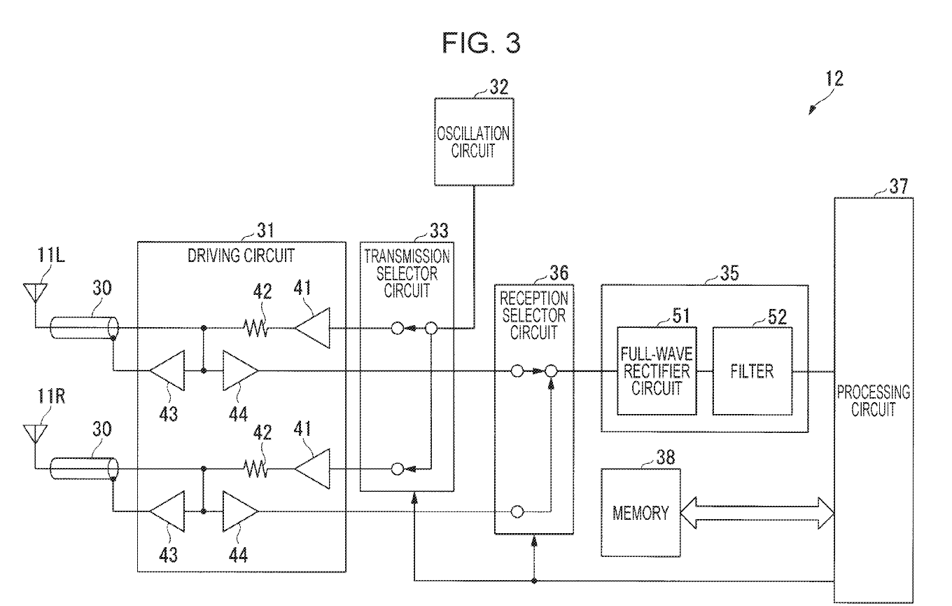

As illustrated in FIG. 3, the heartbeat detection unit 14 includes shield cables 30 each connected to the first sensor (L) 11L and the second sensor (R) 11R, a driving circuit 31, an oscillation circuit 32, a transmission selector circuit 33, a receiving circuit 35, a reception selector circuit 36, a processing circuit 37, and a memory 38.

Each shield cable 30 sheathes lead wires of the first sensor (L) 11L and lead wires of the second sensor (R) 11R. The shield cable 30 is supplied with a shielding current from the driving circuit 31. The shield cables 30 shield the lead wires such that the radio emission power supplied from the driving circuit 31 to respective lead wires of the first sensor (L) 11L and the second sensor (R) 11R is not affected by an external magnetic field and the like.

The driving circuit 31 includes first amplifiers 41, resistors 42, second amplifiers 43, and third amplifiers 44 for the first sensor (L) 11L and the second sensor (R) 11R, respectively.

Each of the first amplifiers 41 is provided between the transmission selector circuit 33 and each of the resistors 42. Each of the first amplifiers 41 amplifies a current supplied from the transmission selector circuit 33 and outputs the amplified current to each of the resistors 42.

Each of the resistors 42 is connected to each of the lead wires for the first sensor (L) 11L and the second sensor (R) 11R and to each of the second amplifiers 43 and each of the third amplifiers 44. Through each of the resistors 42, a current supplied from each of the first amplifiers 41 is divided into and supplied to each of the first sensor (L) 11L and the second sensor (R) 11R and into each of the second amplifiers 43 and each of the third amplifiers 44.

One of the second amplifiers 43 is provided between one of the shield cables 30 and a dividing node between one of the resistors 42 and the first sensor (L) 11L, and the other second amplifier 43 is provided between the other shield cable 30 and a dividing node between the other resistor 42 and the second sensor (R) 11R. Each of the second amplifiers 43 amplifies a current supplied from each of the resistors 42 and supplies the amplified current to each of the shield cables 30 as a shielding current.

One of the third amplifiers 44 is provided between the reception selector circuit 36 and a dividing node between one of the resistors 42 and the first sensor (L) 11L, and the other third amplifier 44 is provided between the reception selector circuit 36 and a dividing node between the other resistor 42 and the second sensor (R) 11R. Each of the third amplifiers 44 amplifies a current supplied from each of the resistors 42 and supplies the amplified current to the reception selector circuit 36.

The oscillation circuit 32 is connected to each of the first amplifiers 41 of the driving circuit 31 via the transmission selector circuit 33. The oscillation circuit 32 outputs a radio frequency signal (radio emission power) at a predetermined frequency to the transmission selector circuit 33.

The transmission selector circuit 33 operates in response to a selection signal input from the processing circuit 37 and supplies radio emission power from the oscillation circuit 32 to each of the two first amplifiers 41 of the driving circuit 31.

The receiving circuit 35 is connected to the third amplifiers 44 of the driving circuit 31 via the reception selection circuit 36. The receiving circuit 35 includes a full-wave rectifier circuit 51 and a filter circuit 52. The full-wave rectifier circuit 51 rectifies the full wave of an output voltage from the reception selector circuit 36 and supplies the rectified voltage to the filter circuit 52. The filter circuit 52 is a smoothing filter circuit, for example, and smooths a signal from the full-wave rectifier 51 to output a signal that is an averaged output of the output voltage of the filter circuit 52. The filter circuit 52 supplies the filtered signal to the processing circuit 37.

The reception selector circuit 36 operates in response to a selection signal input from the processing circuit 37 and supplies signals from the third amplifiers 44 of the driving circuit 31 to the full-wave rectifier circuit 51 of the receiving circuit 35.

The processing circuit 37 compares an averaged output signal of the output voltage supplied from the receiving circuit 35 with a threshold of the output voltage pre-stored in the memory 38 and determines whether or not the electrostatic capacitance detected by each of the first sensor (L) 11L and the second sensor (R) 11R is greater than or equal to a predetermined value. The processing circuit 37 determines whether or not the averaged output from the receiving circuit 35 is smaller than the threshold, for example, to determine whether or not a dielectric material such as in the case of a human body approaches or comes into contact with each of the first sensor (L) 11L and the second sensor (R) 11R. The processing circuit 37 outputs to the gripping state determination unit 15 information about the determination result as to whether or not a dielectric material approaches or comes into contact with each of the first sensor (L) 11L and the second sensor (R) 11R.

When no dielectric material such as in the case of a human body approaches or is in contact with each of the first sensor (L) 11L and the second sensor (R) 11R, a current flowing from the system due to a radio wave emitted from each of the first sensor (L) 11L and the second sensor (R) 11R is relatively small. This results in an increase in a current flowing from the third amplifiers 44 to the receiving circuit 35 and therefore an increase in an averaged output of the output voltage output from the receiving circuit 35.

In contrast, when a dielectric material such as in the case of a human body approaches or is in contact with each of the first sensor (L) 11L and the second sensor (R) 11R, a current flowing from the system due to a radio wave emitted from each of the first sensor (L) 11L and the second sensor (R) 11R is relatively large. This results in a decrease in a current flowing from the third amplifiers 44 to the receiving circuit 35 and therefore a decrease in an averaged output of the output voltage output from the receiving circuit 35.

Accordingly, the memory 38 stores therein data of thresholds for determining an averaged output from the receiving circuit 35 for respective cases where a dielectric material such as in the case of a human body approaches or is in contact with each of the first sensor (L) 11L and the second sensor (R) 11R and where no dielectric material approaches or is in contact with each of the first sensor (L) 11L and the second sensor (R) 11R.

As illustrated in FIG. 2, the first electrode (L) 13L and the second electrode (R) 13R are provided in a left part and a right part, respectively, of the steering wheel 22 in a straight-running position (a neutral position). In the steering wheel 22, the first electrode (L) 13L and the second electrode (R) 13R are provided near to where the first sensor (L) 11L and the second sensor (R) 11R are respectively arranged. The first electrode (L) 13L is provided in the left part of the steering wheel in the neutral position, that is, provided in the portion gripped by the driver's left hand. The second electrode (R) 13R is provided in the right part of the steering wheel in the neutral position, that is, provided in the portion gripped by the driver's right hand. The first electrode (L) and the second electrode (R) 13R are provided in some or all of an inner circumference surface, an outer circumference surface, a side surface, and the like of the steering wheel 22.

The first electrode (L) 13L and the second electrode (R) 13R may be formed of any electro-conductive material. For example, the first electrode (L) 13L and the second electrode (R) 13R are formed by coating the surface of the steering wheel 22 with an electro-conductive material, or by an electro-conductive leather material, or the like.

The first electrode (L) 13L and the second electrode (R) 13R are connected to the heartbeat detection unit 14.

The heartbeat detection unit 14 uses a potential difference between the first electrode (L) 13L and the second electrode (R) 13R to determine whether the steering wheel 22 is gripped by both hands or one hand of the driver. Furthermore, the heartbeat detection unit 14 determines whether or not an electro-conductive foreign material, and not the driver's hand, is in contact with the steering wheel 22.

When the first electrode (L) 13L and the second electrode (R) 13R are contacted by both hands of the driver, the heartbeat detection unit 14 detects the heartbeat of the driver in response to a potential difference between the first electrode (L) 13L and the second electrode (R) 13R satisfying a predetermined condition. When the heartbeat of the driver is detected, the heartbeat detection unit 14 determines that the steering wheel 22 is gripped by both hands of the driver.

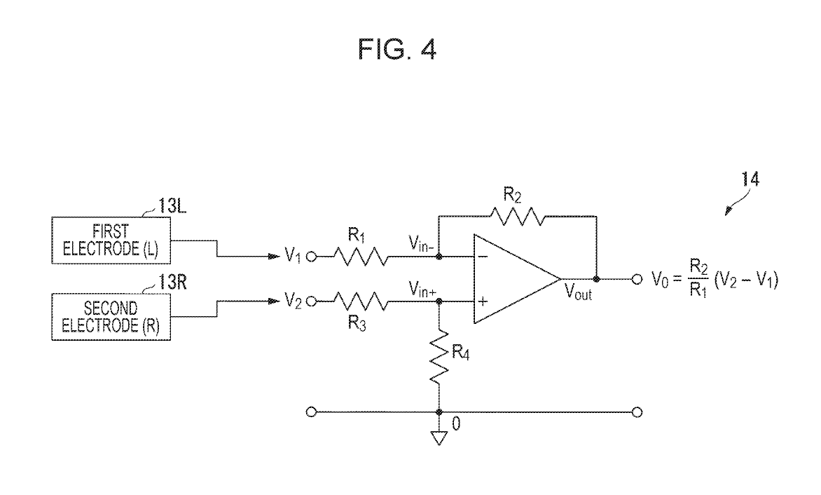

As illustrated in FIG. 4, the heartbeat detection unit 14 includes a differential amplifier circuit. This differential amplifier circuit includes an input resistor R₁ and a feedback resistor R₂ that are connected to an inverting input terminal of an operational amplifier and an input resistor R₃ and a grounding resistor R₄ that are connected to a non-inverting input terminal of the operational amplifier. The input resistors R₁ and R₃ have the same resistance, and the feedback resistor R₂ and the grounding resistor R₄ have the same resistance. A voltage V₀ output from the heartbeat detection unit 14 is expressed by the following equation (1):

V₀=(R₂/R₁)×(V₂−V₁) (1)

where V₁ is the voltage of the first electrode (L) 13L, V₂ is the voltage of the second electrode (R) 13R, R₁ is the resistance of the input resistor R₁, and R₂ is the resistance of the feedback resistor R₂.

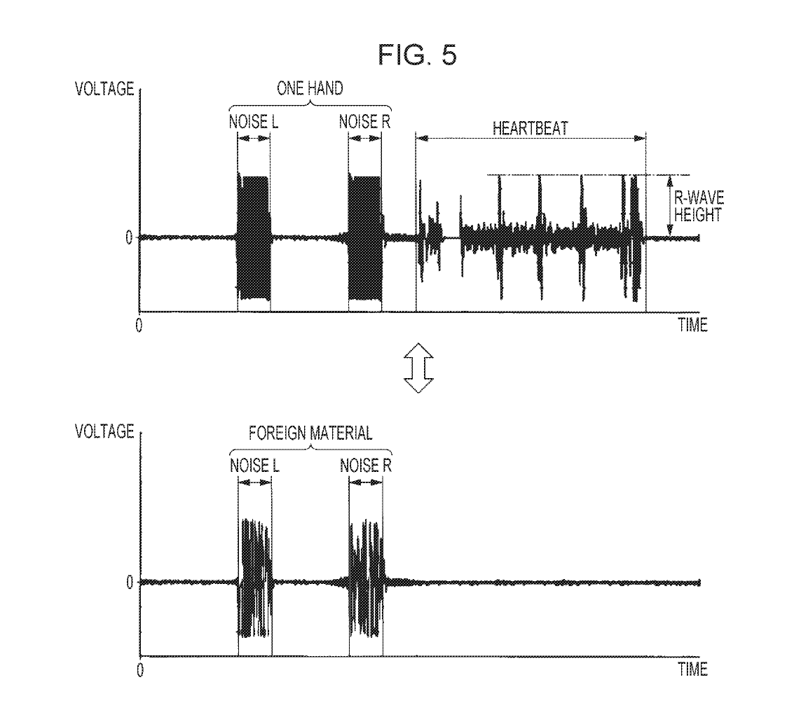

When the steering wheel 22 is gripped by only one hand of the driver, only one of the first electrode (L) 13L and the second electrode (R) 13R is contacted by the driver's hand. Therefore, the heartbeat detection unit 14 outputs noise (L) or noise (R) as illustrated in FIG. 5. For the noise (L), because the voltage V₂ of the second electrode (R) 13R is zero, the voltage of the noise (L) is calculated as (R₂/R₁)×V₁. For the noise (R), because the voltage V₁ of the first electrode (L) 13L is zero, the voltage of the noise (R) is calculated as (R₂/R₁)×V₂.

When the steering wheel 22 is gripped by both hands of the driver, the first electrode (L) 13L and the second electrode (R) 13R are contacted by both hands of the driver. Therefore, in-phase noise from the first electrode (L) 13L and in-phase noise from the second electrode (R) 13R are input to the heartbeat detection unit 14. Thereby, the heartbeat detection unit 14 cancels the noise of the first electrode (L) 13L and the noise of the second electrode (R) 13R and outputs a signal of the heartbeat of the driver in which the signal has a voltage corresponding to an R-wave height according to (R₂/R₁)×(V₂−V₁). Note that a waveform indicating a change in the potential difference between the first electrode (L) 13L and the second electrode (R) 13R is obtained by the limb lead (that is, the first induction (taken between a right wrist and a left wrist in a standard limb lead)), and the R-wave is a wave having the highest amplitude in one cycle in the obtained waveform. The heartbeat detection unit 14 detects a heart rate based on a length of the interval of the R-waves that periodically occur.

When a foreign material (for example, a metal beverage bottle) whose electro-conductivity is less than that of a human body is in contact with the steering wheel 22, the heartbeat detection unit 14 outputs a smaller amount of noise (L) or noise (R) than in the case where one hand of the driver is in contact with the steering wheel 22, as illustrated in FIG. 5.

The gripping state determination unit 15 determines a gripping state of the steering wheel 22 of the driver based on an electrostatic capacitance detected by the electrostatic capacitance detection unit 12 and a voltage pattern of the first electrode (L) 13L and the second electrode (R) 13R detected by the heartbeat detection unit 14.

When an electrostatic capacitance that is greater than or equal to a predetermined value is detected by the electrostatic capacitance detection unit 12 and the heartbeat of the driver is detected by the heartbeat detection unit 14, the gripping state determination unit 15 determines that the driver is gripping the steering wheel 22 with both hands.

When an electrostatic capacitance that is greater than or equal to a predetermined value is detected by the electrostatic capacitance detection unit 12, and no heartbeat of the driver is detected and a noise that is greater than or equal to a predetermined amount is detected in the voltage pattern of the first electrode (L) 13L and the second electrode (R) 13R by the heartbeat detection unit 14, the gripping state determination unit 15 determines that the driver is gripping the steering wheel 22 with one hand.

When an electrostatic capacitance that is greater than or equal to a predetermined value is detected by the electrostatic capacitance detection unit 12, and no heartbeat of the driver is detected and a noise that is greater than or equal to a lower limit threshold and less than the predetermined amount is detected in the voltage pattern of the first electrode (L) 13L and the second electrode (R) 13R by the heartbeat detection unit 14, the gripping state determination unit 15 determines that an electro-conductive foreign material is in contact with the steering wheel 22.

When an electrostatic capacitance that is greater than or equal to a predetermined value is detected by the electrostatic capacitance detection unit 12, and no heartbeat of the driver is detected and a noise that is less than the lower limit threshold is detected in the voltage pattern of the first electrode (L) 13L and the second electrode (R) 13R by the heartbeat detection unit 14, the gripping state determination unit 15 determines that the driver is gripping the steering wheel 22 with one or both of the hands covered by gloves.

The alarming control unit 16 issues various alarming to the driver by using the display 20 and the speaker 21.

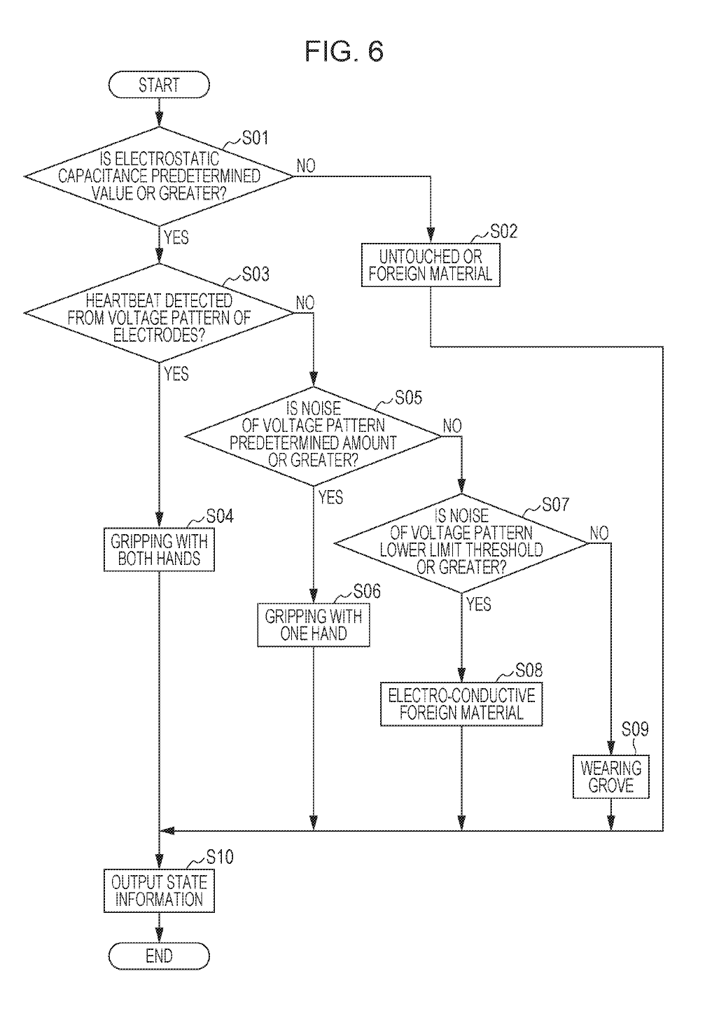

The gripping-detection device 10 according to the present embodiment includes the configuration described above, and operation of the gripping-detection device 10 will then be described with reference to a flowchart illustrated in FIG. 6.

First, the gripping state determination unit 15 determines whether or not an electrostatic capacitance that is greater than or equal to a predetermined value is detected by the electrostatic capacitance detection unit 12 (step S01).

If the determination result is “NO” (step S01: NO), the gripping state determination unit 15 transfers the process to step S02.

On the other hand, if the determination result is “YES” (step S01: YES), the gripping state determination unit 15 transfers the process to step S03.

Then, the gripping state determination unit 15 determines that there is a state where the driver leaves the hands untouched to the steering wheel 22 or a state where a non-dielectric foreign material is in contact with the steering wheel 22 (step S02). The gripping state determination unit 15 then transfers the process to step S10.

Further, the gripping state determination unit 15 determines whether or not the heartbeat of the driver is detected by the heartbeat detection unit 14 based on a voltage pattern of the first electrode (L) 13L and the second electrode (R) 13R (step S03).

If the determination result is “YES” (step S03: YES), the gripping state determination unit 15 transfers the process to step S04.

On the other hand, if the determination result is “NO” (step S03: NO), the gripping state determination unit 15 transfers the process to step S05.

Then, the gripping state determination unit 15 determines that the driver is gripping the steering wheel 22 with both hands (step S04). The gripping state determination unit 15 then transfers the process to step S10.

Further, the gripping state determination unit 15 determines whether or not a noise that is greater than or equal to a predetermined amount is detected in a voltage pattern of the first electrode (L) 13L and the second electrode (R) 13R (step S05).

If the determination result is “YES” (step S05: YES), the gripping state determination unit 15 transfers the process to step S06.

On the other hand, if the determination result is “NO” (step S05: NO), the gripping state determination unit 15 transfers the process to step S07.

Then, the gripping state determination unit 15 determines that the driver is gripping the steering wheel 22 with one hand (step S06). The gripping state determination unit 15 then transfers the process to step S10.

Further, the gripping state determination unit 15 determines whether or not a noise in a voltage pattern of the first electrode (L) 13L and the second electrode (R) 13R is greater than or equal to a lower limit threshold (step S07).

If the determination result is “YES” (step S07: YES), the gripping state determination unit 15 transfers the process to step S08.

On the other hand, if the determination result is “NO” (step S07: NO), the gripping state determination unit 15 transfers the process to step S09.

Then, the gripping state determination unit 15 determines that an electro-conductive foreign material is in contact with the steering wheel 22 (step S08). The gripping state determination unit 15 then transfers the process to step S10.

Further, the gripping state determination unit 15 determines that the driver is gripping the steering wheel 22 with one of both of the hands covered by gloves (step S09). The gripping state determination unit 15 then transfers the process to step S10.

Next, the gripping state determination unit 15 outputs, to an external unit, information of the gripping state of the driver, the contact state of the foreign material on the steering wheel, or the like (step S10). The alarming control unit 16 then ends the process.

As described above, according to the gripping-detection device 10 of the present embodiment, in the steering wheel 22, a gripping state of the hand or hands of the driver can be detected with a high accuracy by detecting an electrostatic capacitance and a voltage pattern of the first electrode (L) 13L and the second electrode (R) 13R.

First, the likelihood of the driver being in contact with the steering wheel 22 can be detected by detecting the electrostatic capacitance. Then, when there is a likelihood that the driver is in contact with the steering wheel 22, particular conditions of the gripping state can be determined based on a voltage pattern of the first electrode (L) 13L and the second electrode (R) 13R. This enables a simplified determination process and therefore faster determination compared to the case where determination is made based on a voltage pattern of the first electrode (L) 13L and the second electrode (R) 13R prior to detection of an electrostatic capacitance, for example.

Furthermore, a state that a foreign material other than the driver's hand is in contact with the steering wheel 22 can be determined by determining whether or not the noise (L) or the noise (R) in a voltage pattern of the first electrode (L) 13L and the second electrode (R) 13R is greater than or equal to a predetermined amount.

Modified examples of the embodiment described above will be described below.

Although, in the embodiment described above, the gripping state determination unit 15 determines that the driver is gripping the steering wheel 22 with one or both of the hands covered by gloves when the noise in the voltage pattern of the first electrode (L) 13L and the second electrode (R) 13R is less than the lower limit threshold, the embodiment is not limited thereto.

The gripping state determination unit 15 may discriminate a state where the driver is gripping the steering wheel 22 with one or both of the bare hands and a state where the driver is gripping the steering wheel 22 with one of both of the hands covered by gloves, based on an electrostatic capacitance detected by the electrostatic capacitance detection unit 12.

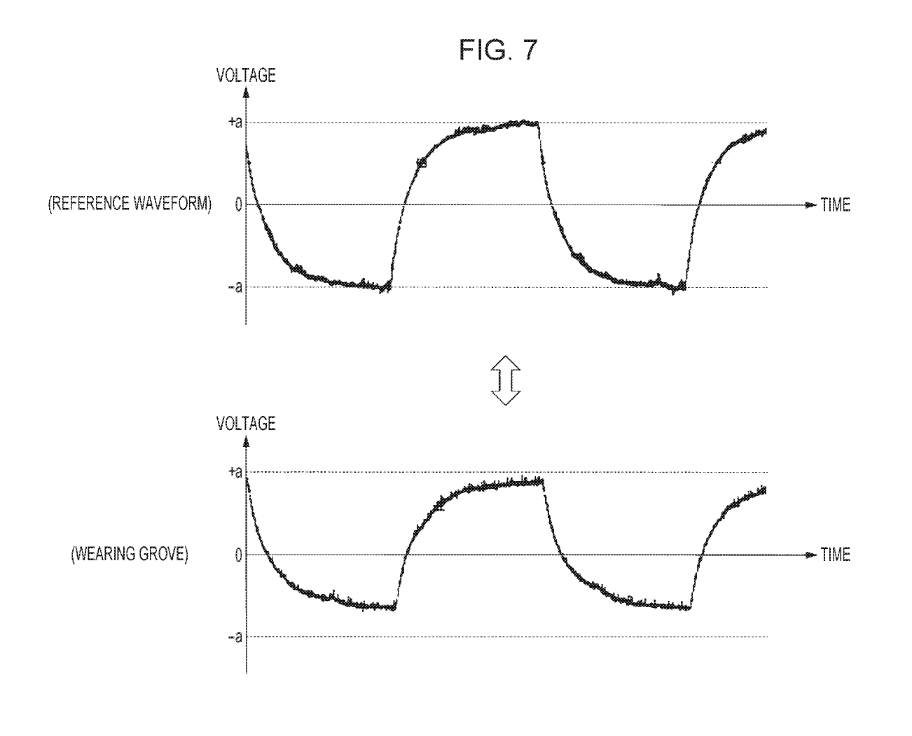

The gripping state determination unit 15 may discriminate these gripping states by using a reduction in the averaged output that varies in accordance with a gripping state of the driver, in which the reduction is a difference from the averaged output that is output from the receiving circuit 35 in a state where a dielectric material such as in the case of a human body neither approaches nor is in contact with the steering wheel 22, for example. As illustrated in FIG. 7, the gripping state determination unit 15 knows in advance each reduction in the amplitude for each of the case where the driver is with bare hands and the case where the driver is wearing gloves based on a reference waveform that is a waveform of the output voltage in a state where no dielectric material approaches or is in contact with the steering wheel 22. The gripping state determination unit 15 determines that the driver is gripping the steering wheel 22 with one or both of the bare hands when a reduction in the amplitude from the reference waveform is greater than or equal to a first threshold, for example. On the other hand, the gripping state determination unit 15 determines that the driver is gripping the steering wheel 22 with one of both of the hands covered by gloves when a reduction in the amplitude from the reference waveform is less than the first threshold.

In the embodiment described above, a part of or all of the electrostatic capacitance detection unit 12, the heartbeat detection unit 14, the gripping state determination unit 15, and the alarming control unit 16 may be a functional unit that is functioned when a CPU (Central Processing Unit) executes a program. Further, these components may be realized as an integrated circuit such as an LSI (Large Scale Integration), and respective functional blocks of these components may be implemented as separate processors, or a part of or all of the functional blocks may be integrated and implemented as a processor. Further, a scheme of integrating circuits is not limited to LSI but may be implemented with a dedicated circuit or a general purpose processor. When a new technology for integrating circuits that can replace LSI emerges in the progress of semiconductor technology, an integrated circuit according to such technology may be used.

The embodiments have been presented by way of example and it is not intended to limit the scope of the present application. These embodiments can be implemented in other various forms, and various omission, replacement, and/or alternation can be made without departing from the spirit of the present disclosure. These embodiments and their modifications are intended to be included in the scope and spirit of the present disclosure and included in the scope of the claimed invention and equivalents thereof.

What is claimed is:

1. A gripping-detection device comprising:
a pair of electrodes provided to a steering wheel of a vehicle, each of the pair of electrodes being arranged on the steering wheel to be in contact with one of driver's hands;
a heartbeat detection unit that detects a heartbeat of a driver that is in contact with the pair of electrodes based on a voltage pattern obtained from the pair of electrodes;
an electrostatic capacitance detection unit that is provided to the steering wheel in a vicinity of the pair of electrodes to detect an electrostatic capacitance; and
a gripping state determination unit configured to determine a gripping state of the driver's hands with respect to the steering wheel based on the electrostatic capacitance detected by the electrostatic capacitance detection unit and a voltage pattern of the pair of electrodes detected by the heartbeat detection unit.

2. The gripping-detection device according to claim 1, wherein the heartbeat detection unit starts the detection of the heartbeat of the driver when the electrostatic capacitance detection unit detects the electrostatic capacitance that is greater than or equal to a predetermined value.

3. The gripping-detection device according to claim 2, wherein, when the electrostatic capacitance detection unit detects the electrostatic capacitance that is greater than or equal to a predetermined value, and the heartbeat of the driver is detected by the heartbeat detection unit, the gripping state determination unit determines that the driver is gripping the steering wheel with both of the hands.

4. The gripping-detection device according to claim 2, wherein, when the electrostatic capacitance detection unit detects the electrostatic capacitance that is greater than or equal to a predetermined value, and the heartbeat detection unit detects no heartbeat of the driver and detects a noise that is greater than or equal to a predetermined amount in the voltage pattern, the gripping state determination unit determines that the driver is gripping the steering wheel with one of the hands.

5. The gripping-detection device according to claim 2, wherein, when the electrostatic capacitance detection unit detects the electrostatic capacitance that is greater than or equal to a predetermined value and the heartbeat detection unit detects no heartbeat of the driver and detects a noise that is greater than or equal to a lower limit threshold and less than a predetermined amount in the voltage pattern, the gripping state determination unit determines that a foreign material is in contact with the steering wheel.

6. The gripping-detection device according to claim 2, wherein, when the electrostatic capacitance detection unit detects the electrostatic capacitance that is greater than or equal to a predetermined value, and the heartbeat detection unit detects no heartbeat of the driver and detects a noise that is less than a lower limit threshold in the voltage pattern, the gripping state determination unit determines that the driver is gripping the steering wheel with one of the hands that is covered by a glove.
